(19) United States
(12) Patent Application Publication
(10) Pub. No.: US 2008/0183054 A1
(43) Pub. Date: Jul. 31, 2008

(54) COMBINATION LEVEL ALARMS AND ALARM PERSISTENCE FOR PATIENT MONITORING

(76) Inventors: PETER KROEGER, OTTAWA (CA); JOHN A. MILLER, OTTAWA (CA)

Correspondence Address: SMART & BIGGAR;P.O. BOX 2999, STATION D, 900-55 METCALFE STREET, OTTAWA, ON, K1P5Y6, omitted

(21) Appl. No.: 12/020,099

(22) Filed: Jan. 25, 2008

Related U.S. Application Data
(60) Provisional application No. 60/887,168, filed on Jan. 30, 2007.

(51) Int. Cl. A61B 5/00

(52) U.S. Cl. 600/301; 600/300

(57) ABSTRACT

Combination level alarms and alarm persistence for patient monitoring, as well as related methods and apparatus, are disclosed. For a combination level alarm, respective values for multiple physiological conditions are received. A determination is made as to whether the received values satisfy respective alarm criteria for the physiological conditions, and if so, an alarm is raised. An alarm persistence feature involves periodically receiving values of a physiological condition, and determining whether the received values have satisfied an alarm criterion for at least a predetermined portion of a predetermined period of time. If so, an alarm is maintained. Combination alarms and alarm persistence may be implemented individually or together.

### CROSS-REFERENCE TO RELATED APPLICATION

This application claims the benefit of U.S. Provisional Patent Application Ser. No. 60/887,168, filed on Jan. 30, 2007, and entitled “Combination Level Alarm with Persistence for Patient Monitoring”, the entire contents of which are incorporated herein by reference.

### FIELD OF THE INVENTION

The present invention relates to managing alarms in physiological monitoring systems.

### BACKGROUND

Medical monitoring devices can be configured to raise an alarm when a vital sign or physiological condition of a patient exceeds an upper and/or lower limit. The level of alarm raised may be Yellow or Red depending on if the limit was exceeded by a small or large degree. The alarm levels may be reflected in different audible notifications and may display in different colours. Different vital signs may be given different priority, which impacts the order of alarm notification. Typically, there is one alarm for each vital sign monitored. Yellow and Red alarms can be configured to be “latching”, so that the alarm remains on until acknowledged. Once an alarm is latched, the alarm will continue regardless of whether the alarm condition continues.

### SUMMARY OF THE INVENTION

In one aspect of the present invention, there is provided a method comprising: periodically receiving values of a physiological condition from a sensor; determining whether the received values have satisfied an alarm criterion for at least a predetermined portion of a predetermined period of time; and maintaining an alarm where the received values have satisfied the alarm criterion for at least the predetermined portion of the predetermined period of time.

Where the received values indicate values of the physiological condition at respective times, determining may involve determining whether at least a minimum number of the received values that indicate values of the physiological condition during a time period equal to the predetermined period of time immediately preceding a time of a most recently received value satisfied the alarm criterion.

The method may also include periodically receiving values of a further physiological condition from a further sensor, in which case determining may further include determining whether the received values of the further physiological condition have satisfied a further alarm criterion for at least the predetermined portion of the predetermined period of time, and maintaining may involve maintaining the alarm where both the received values of the physiological condition have satisfied the alarm criterion for at least the predetermined portion of the predetermined period of time and the received values of the further physiological condition have satisfied the further alarm criterion for at least the predetermined portion of the predetermined period of time.

In some embodiments, maintaining involves maintaining an indication of the alarm in a GUI (Graphical User Interface).

Such a method may be embodied, for example, in instructions stored on a computer readable medium.

In another aspect of the present invention, there is provided apparatus comprising: a sensor interface that enables the apparatus to periodically receive values of a physiological condition from a sensor; and an analysis module, operatively coupled to the sensor interface, that determines whether the received values have satisfied an alarm criterion for at least a predetermined portion of a predetermined period of time, and maintains an alarm where the received values have satisfied the alarm criterion for at least the predetermined portion of the predetermined period of time.

The received values may indicate values of the physiological condition at respective times, and if so, the analysis module may determine whether the received values have satisfied the alarm criterion for at least the predetermined portion of the predetermined period of time by determining whether at least a minimum number of the received values that indicate values of the physiological condition during a time period equal to the predetermined period of time immediately preceding a time of a most recently received value satisfied the alarm criterion.

In some embodiments, the sensor interface further enables the apparatus to periodically receive values of a further physiological condition from a further sensor. The analysis module may then further determines whether the received values of the further physiological condition have satisfied a further alarm criterion for at least the predetermined portion of the predetermined period of time and maintain the alarm where both the received values of the physiological condition have satisfied the alarm criterion for at least the predetermined portion of the predetermined period of time and the received values of the further physiological condition have satisfied the further alarm criterion for at least the predetermined portion of the predetermined period of time.

The apparatus may also include a user interface, operatively coupled to the analysis module, that enables interaction with a user through a GUI, in which case the analysis module may maintain the alarm by maintaining an indication of the alarm in the GUI.

The sensor is located remotely from the apparatus in some embodiments.

A method according to another aspect of the invention comprises: receiving respective values for a plurality of physiological conditions; determining whether the received values satisfy respective alarm criteria for the plurality of physiological conditions; and raising an alarm where the received values satisfy the respective alarm criteria for the plurality of physiological conditions.

The method may also include receiving user inputs for configuring the respective alarm criteria for the plurality of physiological conditions.

In some embodiments, the method includes the further operations of determining whether an individual alarm criterion for one of the plurality of physiological conditions is satisfied by the received value of the one of the physiological conditions; and raising an individual alarm for the one of the physiological conditions where the individual alarm criterion for the one of the physiological conditions is satisfied by the received value of the one of the physiological conditions.

Additional operations provided in some embodiments include receiving a further value for a further physiological condition other than the plurality of physiological conditions; determining whether the received further value satisfies a further alarm criterion for the further physiological condition; and raising an alarm for the further physiological condition where the received further value satisfies the further alarm criterion for the further physiological condition.

As noted above, a method may be embodied in instructions stored on a computer readable medium.

Another aspect of the invention provides apparatus comprising: a sensor interface that enables the apparatus to receive respective values for a plurality of physiological conditions; and an analysis module, operatively coupled to the sensor interface, that determines whether the received values satisfy respective alarm criteria for the plurality of physiological conditions, and raises an alarm where the received values satisfy the respective alarm criteria for the plurality of physiological conditions.

The apparatus may also include a user interface, operatively coupled to the analysis module, that enables the apparatus to receive user inputs for configuring the respective alarm criteria for the plurality of physiological conditions.

In some embodiments, the analysis module further determines whether an individual alarm criterion for one of the plurality of physiological conditions is satisfied by the received value of the one of the physiological conditions, and raises an individual alarm for the one of the physiological conditions where the individual alarm criterion for the one of the physiological conditions is satisfied by the received value of the one of the physiological conditions.

The sensor interface may further enable the apparatus to receive a further value for a further physiological condition other than the plurality of physiological conditions, in which case the analysis module may determine whether the received further value satisfies a further alarm criterion for the further physiological condition, and raise an alarm for the further physiological condition where the received further value satisfies the further alarm criterion for the further physiological condition.

The apparatus may also include a memory for storing the respective alarm criteria for the plurality of physiological conditions. The analysis module may then access the memory to determine whether the received values satisfy the respective alarm criteria for the plurality of physiological conditions.

Other aspects and features of the present invention will become apparent, to those ordinarily skilled in the art, upon review of the following description of specific embodiments of the invention.

### BRIEF DESCRIPTION OF THE DRAWINGS

Examples of embodiments of the invention will now be described in greater detail with reference to the accompanying drawings, in which:

FIG. 1 is an overview diagram of a system in which embodiments of the present invention could be used;

FIG. 2 is a diagram of another system in which embodiments of the present invention could be used;

FIG. 3 is a diagram of a system architecture in which embodiments of the present invention could be used;

FIG. 4 is a comparison chart comparing a method according to an embodiment of the present invention to a conventional method; and

FIG. 5 is a comparison chart comparing a method according to an embodiment of the present invention to a conventional method.

FIG. 6 is a block diagram of an example PMU (Patient Monitoring Unit).

FIG. 7 is a block diagram of an example patient monitoring server.

FIG. 8 is a block diagram of an example CMP (Clinical Monitoring Position).

FIG. 9 is a block diagram of an apparatus according to an embodiment of the invention.

### DETAILED DESCRIPTION OF THE PREFERRED EMBODIMENTS

Embodiments of the present invention include methods and systems for raising alarms in response to monitored physiological conditions. In some embodiments, alarms are raised if a monitored physiological condition satisfies an alarm criterion for a minimum portion of a predetermined amount of time. In other embodiments, an alarm is raised if a combination of predetermined criteria is satisfied. Some embodiments of the invention are referred to herein as Combination Level Alarm with Persistence detection (CLAP).

### Physiology Monitoring

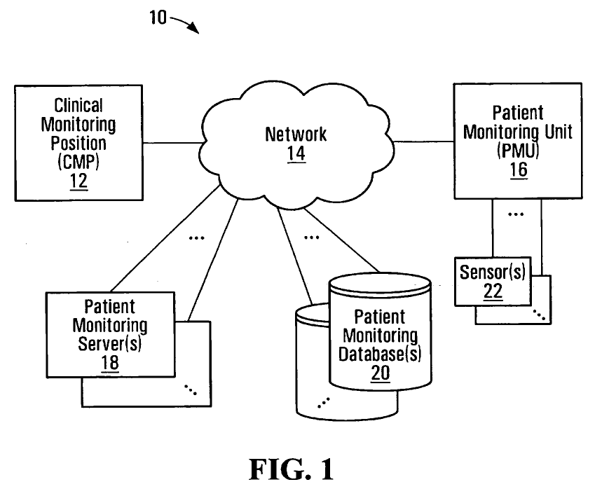

A PM (Physiology Monitoring) system 10 in which the present invention may be used, as depicted in FIG. 1, comprises a PMU (Patient Monitoring Unit) 16 for monitoring physiological conditions of a patient through one or more sensors 22, a CMP (Clinical Monitoring Position) 12 for displaying monitoring results from the PMU 16, one or more databases 20 for storing the monitoring results from the PMU 16, one or more servers 18 for processing and forwarding monitoring results from the PMU 16, and a communication network 14, illustratively an IP network. The PMU 16 and the CMP 12 communicate with each other over the network 14.

It should be appreciated that a system in which embodiments of the present invention is implemented may include other components than those shown in FIG. 1. For example, where the network 14 is a public network, there will typically be other users that communicate through the network for other purposes than physiological monitoring.

Such users have not been shown in FIG. 1 in order to avoid overly complicating the drawing. Thus, more generally, embodiments of the invention may include further, fewer, or different elements interconnected in a similar or different manner than explicitly shown. The contents of FIG. 1, as well as the other drawings, are intended solely for the purposes of illustration.

The PMU 16, as shown, comprises or is at least operatively coupled to one or more sensors 22 for sensing the physiological conditions of a patient. The PMU 16 is also operatively coupled to the network 14, through a wireless link in some embodiments. The sensor(s) 22 may include any or all of the following, but are not in any way limited thereto: temperature sensors, EKG sensors, ECG sensors, heart rate sensors, water sensors, electrolyte sensors, blood pressure sensors, oxygen saturation (SpO2) sensors, and hydration sensors. These and other types of sensors are available from various manufacturers. The PMU 16 and sensor(s) 22 may be part of a device that is wearable by a patient, although other implementations are also possible.

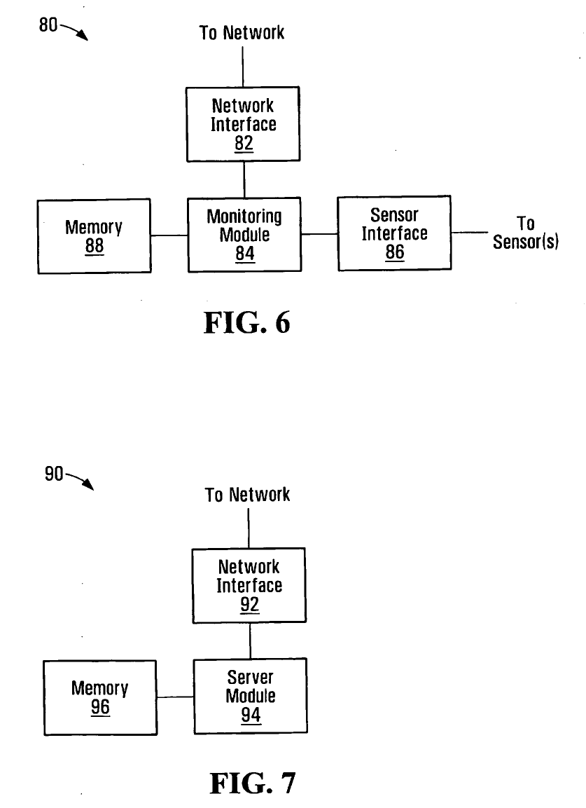

Communication with the network 14 may, for example, be through a wireless link such as a General Packet Radio Service (GPRS) radio link or a WiFi link. In some embodiments, satellite communication is used. An example PMU is described in further detail below with reference to FIG. 6.

Many different types of communication networks that could serve as the network 14 are available, and will be familiar to those skilled in the art. Public networks such as the Internet, private networks, combinations of different types of networks, etc., are all contemplated.

A patient monitoring server 18, although shown in FIG. 1 separately from the database(s) 20, may include memory for storing patient monitoring information. A server 18 would also include a network interface, as well as components implementing server functions, in the example system 10. An example of a server 18 is described below with reference to FIG. 7. Since the server(s) 18 and the database(s) 20 are operatively coupled to the network 14, they may be physically deployed at any location from which the network 14 is accessible. Multiple servers 18 may be deployed at different locations, and multiple databases 20 may similarly be deployed at different locations. It should also be appreciated that a server 18 and a database 20 need not necessarily be co-located.

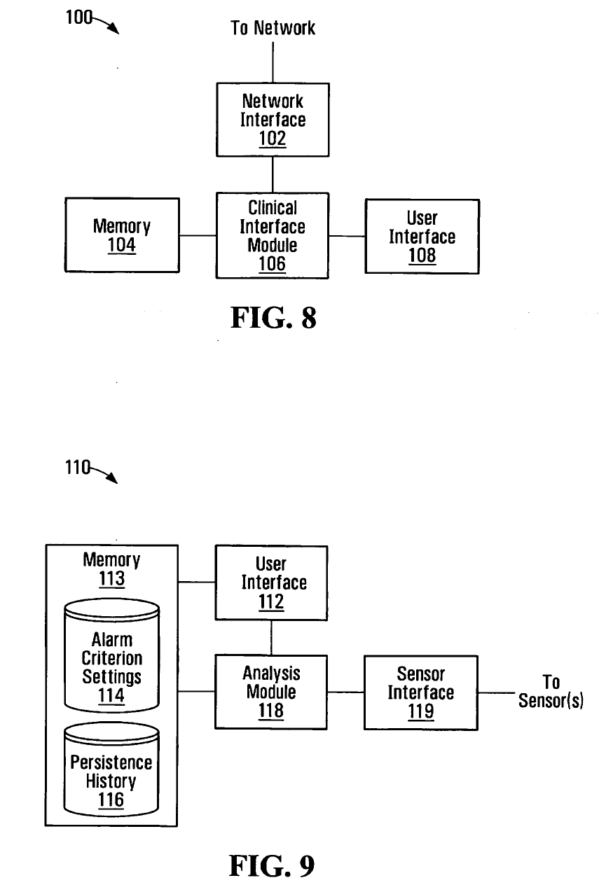

The CMP 12 is operatively coupled to the network 14, and can thus connect to the server(s) 18 through a communication link such as an Ethernet link of any kind, for example. The CMP 12 allows patient monitoring information to be reviewed remotely in the example shown, although as described below, remote monitoring is one optional feature that need not be provided in all embodiments of the present invention. An example CMP is described below with reference to FIG. 8.

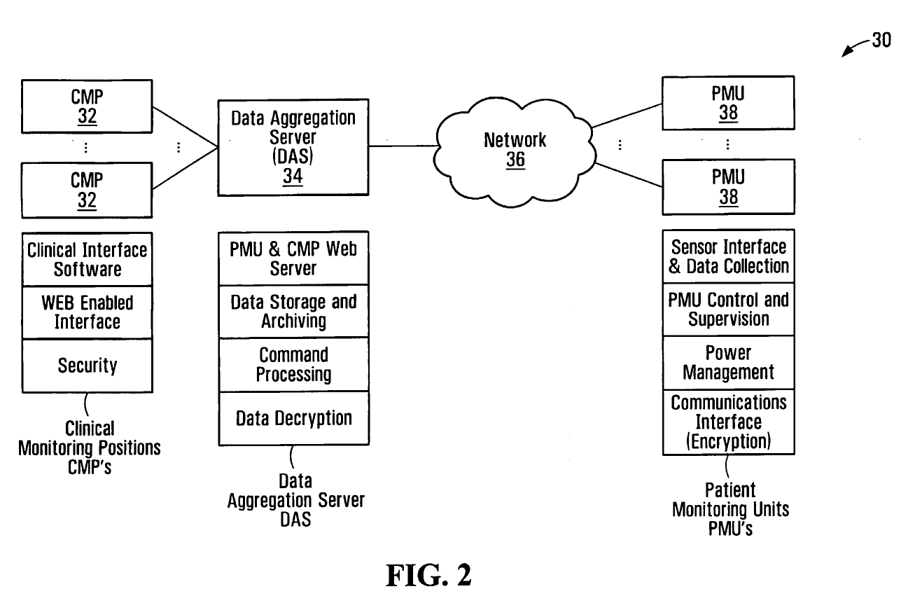

Another example of a PM system 30 is shown in FIG. 2. In the system 30, there are multiple PMUs 38, a private or public network 36, illustratively an IP network, a DAS (Data Aggregation Server) 34, and multiple CMPs 32.

Each of the PMUs 38 may include a sensor interface and data collection module, a PMU control and supervision module, a power management module, and a communications interface, as shown. A wireless LAN (Local Area Network) GPRS Radio may also be provided where the communications interface supports GPRS communications. The communications interface performs encryption of data collected by the PMU in some embodiments. The sensors themselves have not been shown in FIG. 2 in order to avoid overly complicating the drawing, but would be incorporated into or at least operatively coupled to the PMUs 38.

The DAS 34, as shown, includes a PMU and CMP Web Server, a data storage and archiving module, a command processing module, and a data decryption module.

Each CMP 32 comprises, in the example shown, clinical interface software, a web-enabled interface, and a security module. The clinical interface software may be programmed to implement alarms for notifying clinical personnel of various physiological states of monitored patients according to embodiments of the present invention.

A PM system such as shown in FIGS. 1 and 2 could be used in many different applications. Military applications include monitoring patients in field hospitals or triage, monitoring deployed personnel, monitoring of injured soldiers during recovery, and training and rehabilitation of patients released from hospital. Health care applications include expanding ICU (Intensive Care Unit) capacity, research such as drug trials, chronic patient care, acute patient care, home care, and consumer self monitoring. A PM system could also be used by first responders in situations such as terrorist attack, natural disaster, fire fighting, and CBRN (Chemical, Biological, Radiation, Nuclear) events. Another possible use for this type of system would be in managed care situations such as rehabilitation, senior's residence and step-down facilities.

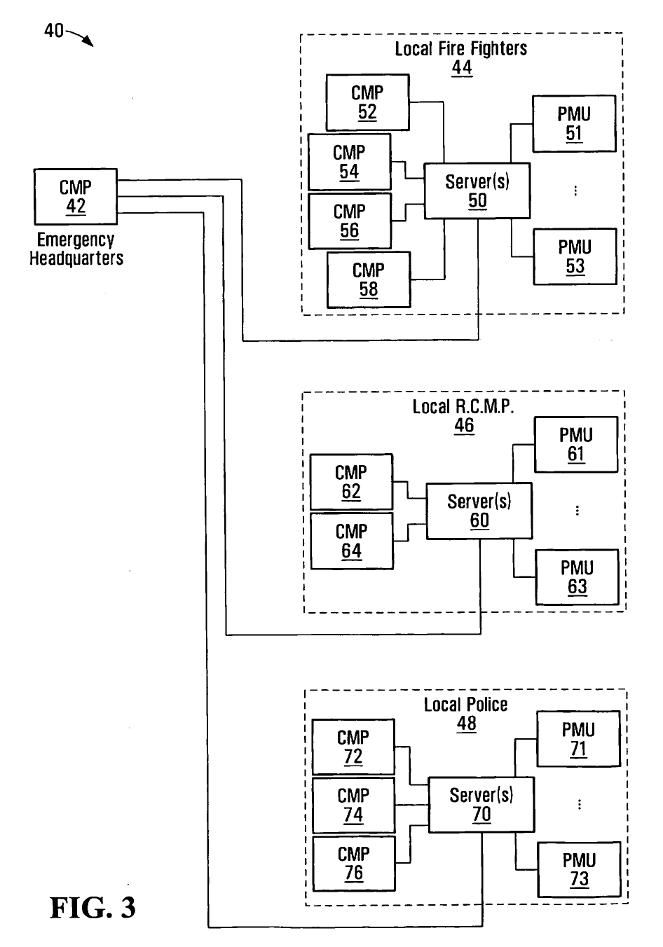

An example embodiment of a network architecture for a PM system 40 according to one embodiment of the invention is depicted in FIG. 3. In the system 40, a CMP 42 at an Emergency HQ (Headquarters) site displays data from three different DASs 50, 60, 70. Each DAS 50, 60, 70 is part of a separate PM system, one for the Local fire fighters 44, one for the local RCMP 46 and one for the local police 48. Each separate PM system 44, 46, 48 comprises a plurality of CMPs 52/54/56/58, 62/64, 72/74/76, a DAS 50, 60, 70 and a plurality of PMUs 51 . . . 53, 61 . . . 63, 71 . . . 73. Communications between the CMP 42 and the DASs 50, 60, 70 may be through a network, for example. In some embodiments, all of the PM systems use an IP network, such as the Internet, for communication.

While the system 40 of FIG. 3 has three PM systems 44, 46, 48, it is to be understood that in other embodiments, any number of systems is possible.

### Alarm Functions

A PM system according to embodiments of the present invention also supports alarm functions, which may generally be intended to accomplish one or more of the following goals:
Alert medical staff of patient(s) in an alarm state in a way that conforms to industry standards and captures their attention as appropriate to the severity of the alarm state;
Reduce the likelihood of false alarms and/or provide some indication that an alarm may be false; and
Alert staff of system related issues (alerts) that impact the ability of the PM system to monitor patient(s).

In one embodiment, the PM system uses the following alarm levels for individual patients:
No Alarm—the patient and PMU monitoring the patient are without alarm;
Yellow System—indicates an alarm with the Patient's PMU (equipment) or ability to be monitored;
Amber (4 levels):
   Amber Latching—no active alarm but a past Red alarm was not acknowledged;
   Amber (Active)—an Amber alarm condition is in effect;
   Amber Recent Persistent—an Amber alarm condition exists for N/M seconds but not currently;
   Amber (Active) Persistent—an Amber alarm condition exists for N/M seconds and currently; and
Red (Active)—a Red alarm condition is in effect.

At any given time, the alarm colour used could be the highest one of the alarms in effect for that patient. The notification of the alarm state is configurable and varies for the level of alarms, in some embodiments.

Having multiple alarm levels (illustratively Amber vs. Red alarms) allows medical personal to respond accordingly. In general, Red alarms require immediate attention and are less likely to be false alarms. In some embodiments, Red alarms also require acknowledgement as described below.

### Latching Alarms

An alarm is said to be “latching” when a notification persists even if the alarm conditions are in effect for only a short period of time. The term “sticky” is also used to describe this feature in some documents and code.

In some embodiments of the present invention, specific alarm levels are configured system wide to be latching or not. A default configuration might apply this feature to Red alarms only, for instance. Some installations may wish to apply this feature to Amber-Persistent or even Amber alarms.

When a latching alarm is first raised, it is given a latching flag. The notification for the alarm will be displayed as long as this flag is set and/or the alarm condition is in effect. The flag is removed when the alarm is acknowledged. The Amber Latching alarm replaces an unacknowledged latching alarm when the alarm conditions are no longer in effect.

Making Red alarms latching ensures that someone is always made aware of a Red alarm event in the case where no one was available to take note at the actual time of occurrence.

### Persistent Alarms

A patient may be in an alarm state continuously, intermittently or as a single occurrence. In the case where the alarm state fluctuates between active and inactive, an embodiment of the PM system detects whether, within the last M seconds, the alarm state is active for at least N seconds. In this case the alarm is considered persistent and will be considered active even for those brief intervals when it would otherwise be inactive.

One implementation of persistent alarm detection uses a data structure having an ordered number of flags equal to the number of seconds in the predetermined period. As time passes, the last flag is discarded and all flags are shifted such that the second to last flag becomes that last and the first flag is in an unknown state ready to be set to indicate the current alarm state. At the same time, a count of all flags is maintained such that it is decremented when the discarded flag is true and incremented when the added flag is true. At any given time, the persistent alarm state is active when the count is equal to or greater than a predetermined number, which corresponds to M seconds in the above example. Another possible implementation omits the use of a count when the count of all set flags may be readily determined directly from the data structure when required for comparison with the predetermined number or persistence time period.

Persistent alarms reduce alarm flicker both visually and in historical data. They also allow medical observers to identify and respond accordingly to a generally continuous alarm condition versus a brief abnormality which might be a false alarm.

### Patient Sensor Alarms

Sensors report vital signs (heart rate, temperature, etc.) with some numeric value. Each numeric value may be assigned an individual upper and lower limit beyond which the PM system considers the respective vital sign to be in an Amber alarm state. Extended upper and lower limits may be set beyond which the PM system considers that vital sign to be in a Red alarm state. These limits might be configured on a per patient basis, for instance. In some embodiments, the PM system retains a “Default” alarm configuration that can easily be assigned to new patients or restored to existing patients.

One implementation of a sensor alarm uses a data structure having minimum and maximum limits for amber and red alarms, as well as alarm state information including alarm level and latching status. Upon a new sensor value arriving, the new value is compared against the various limits and the alarm level set, illustratively to the highest alarm level applicable. When the alarm level is configured to be latching and that alarm level occurs, then a latching status is set.

### Enabling and Disabling Sensor Alarms

For each patient, in some embodiments, each vital sign alarm, also referred to herein as a sensor alarm, may be enabled or disabled. Disabling a sensor alarm may be appropriate to reduce false alarms when that sensor is not completely reliable or the vital sign is not relevant for a particular patient. In some embodiments, when a sensor alarm is disabled, alarm levels in a real time display are not visible and the display indicates that the alarm has been disabled. One example of such an indication is an alarm bell symbol that is grey, with a red circle having a slash drawn over it.

### NIBP (Non-Invasive Blood Pressure) Alarms

In some embodiments of the PM system, NIBP vitals have separate alarm levels and settings for each of systolic, diastolic and average. Since the NIBP is typically not collected in a continuous manner, in some embodiments, any active alarm will expire after a predetermined number of seconds.

### Combination Alarms

In addition to individual sensor alarms, in some embodiments, each patient can be configured with combination alarms. These include alarm limits for a subset of vital signs of which all must be in an alarm state for the combination alarm to be in an alarm state. If less than all participating sensors are in alarm state then no alarm state exists for the combination alarm and the overall patient alarm state is unaffected by that combination alarm.

One implementation of combination alarm detection uses a data structure having the latest alarm state recorded for all sensors included as part of the combination and the alarm state for the combination alarm itself. Upon a new sensor value arriving, the alarm state for the individual sensor is updated and the alarm state for the combination alarm is revised as follows. In one embodiment, the combination alarm state is first assumed to be amber. For each sensor within the combination, if the sensor is not in an alarm state, then the combination alarm is also not in an alarm state and no further sensors need be considered. When all participating sensors are in an alarm state, the alarm state of the combination alarm is set to be the most significant alarm state encountered, in some embodiments.

### ECG (Electro-Cardiogram) Analysis Alarms

In some embodiments, a PM system can raise alarms based on analysis of an ECG signal. Some embodiments of the PM system are able to detect the possibility of fibrillation and raise a Red alarm in this case. The threshold for a fibrillation alarm detector, like other thresholds, may be configurable.

### System Alarms

Some embodiments of the PM also support system alarms.

A first type of system alarm concerns the ability of the PM system to monitor a specific patient and a second concerns the PM system itself.

In some embodiments, the PM system will raise a Yellow alarm for a patient when:
The patient's PMU has not communicated with the DAS within a specific time period;
The battery on the PMU is low on power; and
Some other technical problem exists with the PMU or its sensor(s)

In some embodiments, the PM system itself may raise a “System Alert” alarm when:
The DAS has detected it is low on hard disk space for the database;
The DAS is configured to have a backup DAS but the backup is not operating; and
The DAS is configured to have a minimum number of CMPs connected and there are less than that number.

### Suspended Alarms

For any number of reasons a patient may purposely have the PMU and/or its sensor(s) temporarily removed or be briefly put into a known state such that the sensors would emit readings that would cause an alarm when none is warranted. To avoid false alarms during these periods, some embodiments of the PM system allow alarms to be suspended for a time and restored later.

### Priority of Alarms

A PM system might normally display all active alarms for a given patient on CMPs that monitor that patient. Some CMPs may be in a mode where all (and only) patients in alarm states are listed with all active alarm details. In some embodiments, the priority of an alarm impacts the visible order in which alarm details are displayed. This is in contrast to systems which pop-up each individual alarm in sequence according to priority. For example, in some embodiments, the alarms may be given the following priority, from highest to lowest:
1. Fibrillation (from ECG Analysis)
2. Heart Rate (from ECG)
3. Blood Oxygen (from SpO2)
4. Respiration (likely derived from ECG signal)
5. Blood Pressure (from intermittent NIBP)
6. Temperature
7. Yellow System Alarms.

### Patient Alarm Notification

In some embodiments, when a patient is in an alarm state all CMPs are notified. In one example, a button in the system bar of every CMP switches to “Patient Alarm” mode. This button flashes and indicates the number of patients within the PM system that are in an Alarm state. The “Patient Alarm” mode might list all these patients and their alarm state and details. In addition, in some embodiments, a “Patient Summary” mode of a CMP also includes the patient alarm level as part of the data available.

In some embodiments, the PM system allows CMPs and patients to be part of a named group. In this case, only those CMPs that are part of a patient's group will be notified if the patient is in an alarm state.

Individual CMPs may be configured to have an audible alarm when at least one patient is in alarm state within the PM system. In some embodiments, for those patients that have been selected for monitoring by a CMP, a real time view, in a Multi-patient layout in a GUI for instance, includes an area used to display alarms with a background colour indicating the alarm level. This alarm message might flash (for example, text colour may alternate between white and black) for alarms of Amber (Active) or above. Once an alarm is acknowledged, this message might no longer be displayed except for the Patient Alarm mode. Selecting a patient from this mode could reset the acknowledge state of the alarm so that the alarm message is once again visible in the real time view.

In some embodiments, any sensor in an alarm state will have its vital sign value alternate between two colors having relatively low contrast from each other as a subtler reminder of this state. In addition, an alarm bell symbol in some embodiments will also blink on and off.

Some embodiments may sound an audible alarm regardless of the view. A different sound can be used for Amber vs Red alarms. This may be muted for a predetermined time period, for example for 3 minutes, on individual CMPs for specific patients using the mute button. In other embodiments, there is also a mute button for all patients on an individual CMP. After the predetermined time period, the mute expires. Muting an alarm has no impact on patients' alarm states in the PM system. A CMP may be configured to have no audible notification in general.

### Other Notifications

Some embodiments of the PM system support the ability for the DAS to send email, text message, pager or fax notifications for some alarms.

### System Alarm Notification

In some embodiments, system alarms are displayed in a pop-up panel that extends from a system bar in the CMP. System alarms may produce an audible alert distinct from other audible alarms in order to provide for alarm type differentiation.

### Historic Alarm Indication

In some embodiments, trends data for specific vital signs are stored, including when an alarm was in effect.

A patient notes log is another form of historical information, which may include entries for alarm configuration changes for a patient.

### Combination Alarm Methods:

In addition to alarm conditions for individual vital signs, embodiments of the present invention provide for configuration of combination alarms where each participating vital sign is given respective levels which all must be exceeded for the combination alarm to be raised.

For example, alarms may be configured to occur when:
1. Heart rate exceeds 120 or SpO2 is less than 90 (individual settings);
OR
2. Heart rate exceeds 100 and SpO2 is less than 95 (combination settings).

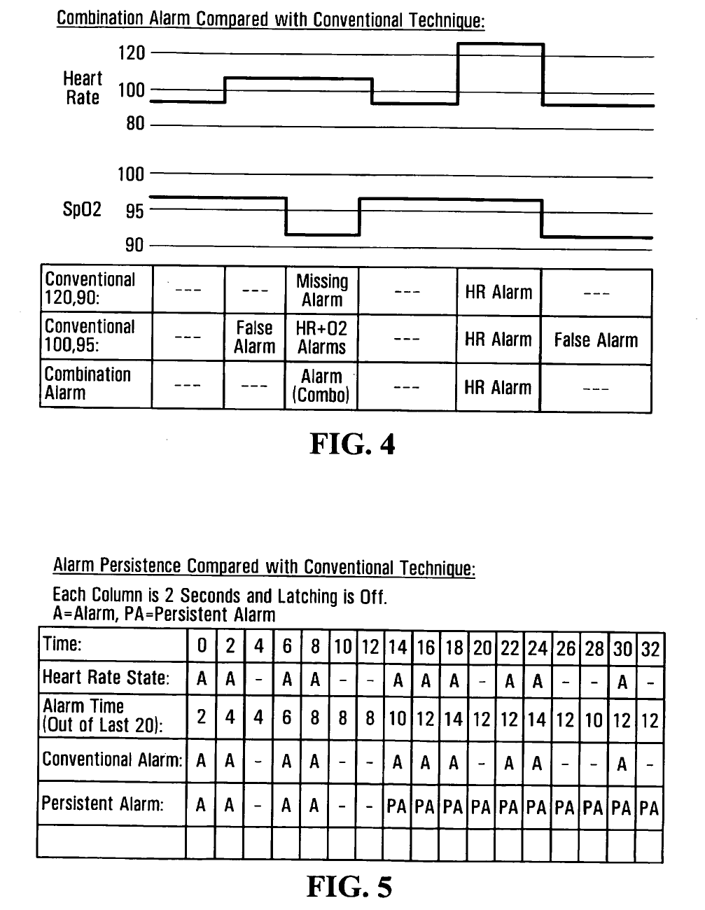

With conventional techniques, the Heart rate and SpO2 limits must be set to 100 and 95, which would cause false alarms assuming each condition is acceptable on its own, or to 120 and 90, which would omit the alarm condition of Heart rate>100 and SpO2<95. FIG. 4 illustrates a comparison of the conventional technique with this combination alarm technique.

In some embodiments, the combination alarm is set independently of individual alarms, including the enabling or disabling of those alarms.

Thus, a method according to one embodiment of the invention might involve receiving respective values for multiple physiological conditions, which would be heart rate and SpO2 in the above example. A determination is then made as to whether the received values satisfy respective alarm criteria for the physiological conditions. An alarm is raised where the received values satisfy the respective alarm criteria.

Any or all of the alarm criteria may be configurable, and accordingly the method may also involve receiving user inputs for configuring the respective alarm criteria.

Individual alarms are also supported in some embodiments. In this case, a further determination is made, as to whether an individual alarm criterion for one of the physiological conditions is satisfied by the received value of that physiological condition, and if so, an individual alarm for that physiological condition is raised.

It should be noted that not all received readings need necessarily be used for the purposes of a combination alarm. A value for another physiological condition, which is not one of the multiple physiological conditions included in the combination alarm, might also be received. If the received value satisfies an alarm criterion for the other physiological condition, then an alarm for the further physiological condition is raised.

Thus, a received sensor reading might be used in combination alarm processing, individual vital or sensor alarm processing, or both.

### Persistent Alarm Detection Methods:

In another embodiment of the present invention, the persistence of each vital sign alarm is tracked so that those vital signs that raise an alarm for N or more of the last M seconds are considered persistent. Unlike a latching mechanism on its own, the persistence property of an alarm allows the alarm to be given a higher priority and different behaviour than a brief non-persistent alarm.

For example, assuming N=10 and M=20, if the heart rate exceeds the limit for a period of time, such as 5 seconds, and then falls below the pre-set threshold, the conventional and proposed techniques would issue a 5 second or latched alarm if latching was set. If the heart rate exceeds the limit for total of 15 seconds out of the previous 20 seconds, then the conventional technique would issue multiple alarms having a total duration of 15 seconds or a latched alarm if latching was set. The proposed technique would issue some brief alarms, until the persistence threshold of 10 is reached, followed by a persistent alarm which would remain on even when the heart rate does not exceed the limit, as long as the persistence criterion is met. The persistent alarm gives more visual information and may be independently configured to be latched or not.

FIG. 5 provides a comparison of the persistence alarm technique of an embodiment of the present invention with the conventional alarm techniques.

A method relating to alarm persistence might thus include periodically receiving values of a physiological condition from a sensor, a heart rate sensor in the above example, and determining whether the received values have satisfied an alarm criterion for at least a predetermined portion of a predetermined period of time. In the above example, the predetermined portion, which is configurable in some embodiments is 10 seconds, and the predetermined period of time is 20 seconds. An alarm is maintained where the received values have satisfied the alarm criterion for at least the predetermined portion of the predetermined period of time.

The alarm may be maintained by maintaining an indication of the alarm in a GUI (Graphical User Interface), for example. The operation of maintaining an alarm may actually be an “active” or “passive” operation. Changing an alarm indication to reflect the fact that an alarm is a persistent alarm is an example of an active operation to maintain an alarm. Where an alarm is already a persistent alarm, maintaining the alarm might involve not taking any action to remove the alarm, i.e., a passive operation.

From FIG. 5, it will be apparent that the received values may indicate values of the physiological condition at respective times. The determining operation may then involve determining whether at least a minimum number of the received values that indicate values of the physiological condition during a time period, which is equal to the predetermined period of time immediately preceding a time of a most recently received value, satisfied the alarm criterion.

Alarm persistence and combination alarms are combined in some embodiments, to allow combination alarms to be persisted. If values of a further physiological condition are periodically received from a further sensor, the determining may include determining whether the received values of the further physiological condition have satisfied a further alarm criterion for at least the predetermined portion of the predetermined period of time. An alarm may then be maintained where both the received values of the physiological condition have satisfied the alarm criterion for at least the predetermined portion of the predetermined period of time and the received values of the further physiological condition have satisfied the further alarm criterion for at least the predetermined portion of the predetermined period of time.

### Implementation

Embodiments of the present invention are generally applicable to all vital signs monitoring equipment. There may be particular applicability to remote monitoring situations where the cost of responding to false alarms can be prohibitively expensive.

The methods described herein may be implemented by hardware, software, firmware or combinations thereof. Computer readable instructions for implementing the methods may be stored on a central server such as a DAS in a monitoring system, on computers at display consoles, such as those at a CMP, or even at patient locations, in PMUs for instance.

Display consoles on which alarms are shown may also show present conditions for one or more patients. The consoles can comprise a central console at a CMP for displaying conditions of many patients or smaller devices such as PDAs (Personal Digital Assistants) carried by medical personnel for displaying conditions of a smaller number of patients.

Considering the issue of possible implementations in further detail, FIGS. 6 to 9 show examples of various components of a PM system.

FIG. 6 is a block diagram of an example PMU 80, which includes one or more network interfaces generally shown at 82, a memory 88, a monitoring module 84 operatively coupled to the network interface and to the memory, and one or more sensor interfaces generally shown at 86 operatively coupled to the monitoring module 84. Other components may also be provided in equipment in or in conjunction with which a PMU is implemented. A PMU might include a battery and a power management module, for example. Additional components would also be present when a PMU is implemented using a patient's PC for instance.

A network interface 82 may include a physical interface and associated communication components, such as a radio for wireless communications, that enable the PMU 80 to communicate with other components of a PM system through a network. Communications, or at least sensor reading and/or patient information, are encrypted in some embodiments. Different network interfaces 82 may be provided to enable communication in different networks or using different protocols, for example.

The sensor interface 86 similarly includes some sort of physical interface and associated components that enable the PMU to receive readings from any of various sensors. Respective sensor interfaces may be provided, for instance, to support interaction with different types of sensors or to allow specific interfaces to be dedicated to particular sensors.

The monitoring module 84 is implemented using a processing element and software stored in the memory 88 in some embodiments. This module at least collects readings from one or more sensors through the sensor interface 86. Processing elements such as microprocessors, microcontrollers, ASICs (Application Specific Integrated Circuits), FPGAs (Field Programmable Gate Arrays), PLDs (Programmable Logic Devices) might be suitable for this purpose. The monitoring module 84 might also support such functions as transmitting sensor readings and patient information through the network interface 82 to a DAS and/or to a CMP, storing sensor readings in the memory 88, and possibly even analysis of sensor readings.

The memory 88 may be implemented using one or more memory devices. Solid state memory devices are common in electronic equipment, although other types of memory devices using movable or even removable storage media may also be used. Software implementing the monitoring module 84, information associated with a patient being monitored, collected sensor readings, and/or possibly other information may be stored in the memory 88.

FIG. 7 is a block diagram of an example server 90, which might be a DAS or other server in a PM system. The server 90 includes one or more network interfaces 92, a memory 96, and a server module 94 operatively coupled to the network interface and to the memory. Although other components may be provided in equipment in or in conjunction with which a server is implemented, those components have not been explicitly shown in FIG. 7 to avoid overly complicating the drawing. For example, a DAS might include, in addition to a server module 94, separate data storage/archiving, command processing, and data encryption/decryption modules. These and other functions may instead be integrated with server functions into the server module 94.

The network interface 92, like the network interface of the PMU 82 (FIG. 6) may include a physical interface and associated communication components that enable the server 90 to communicate with other components of a PM system through a network. A single network interface could potentially be used for all communications between the server 90 and other components of a PM system, although multiple interfaces of different types may be provided in some embodiments.

The server module 94 provides server-side functions for at least managing monitoring data, and may be implemented using a processing element and software stored in the memory 96, for example. Server-side functions may include storing sensor readings received from PMUs to the memory 96, and providing sensor readings from the memory 96 to CMPs, periodically and/or in response to CMP requests. The server module 94 might also incorporate such functions as processing other types of commands and data security (encryption/decryption). These and/or other additional functions may instead be supported using different modules, such as different software modules.

The memory 96 may include one or more memory devices, such as solid state memory devices and/or other types of memory devices. In a data server, memory devices such as disk drives, which use movable storage media, are commonly used to provide relatively large storage capacities. Software implementing the server module 94, information associated with monitored patients being monitored, collected sensor readings, and/or possibly other information may be stored in the memory 96.

FIG. 8 is a block diagram of an example CMP 100, which includes one or more network interfaces 102, a memory 104, a clinical interface module 106 operatively coupled to the network interface(s) and to the memory, and one or more user interfaces 108 operatively coupled to the clinical interface module 106. As noted above for the example PMU 80 and the example server 90 in FIGS. 6 and 7, other components may be provided in equipment in or in conjunction with which a CMP is implemented.

The network interface 102, or each interface where more than one is provided, may include a physical interface and associated communication components. The exact form of a physical interface and its associated components will vary depending on the type(s) of communications to be supported.

The user interface 108 may include one or multiple user interface devices. In one embodiment, a CMP includes a keyboard, a mouse, and a monitor. Information regarding alarms and alerts is displayed to a user on the monitor, and the user inputs information, to set thresholds, to select patients, and/or to acknowledge alarms for instance, using the keyboard and mouse. Presentation and collection of information is enabled through a GUI in some embodiments.

The clinical interface module 106 is implemented using a processing element and software stored in the memory 104 in some embodiments. In a remote monitoring system, the clinical interface module 106 may be responsible for determining whether or not any alarms are to be raised and, if so, raising those alarms through the user interface 108 and/or possibly the network interface 102. Some alarms might be both presented at the CPM 100 and also sent by e-mail through the network interface 102, for instance. Where sensor readings upon which alarm determinations are to be based are encrypted, the clinical interface module 106 or another element provides a decryption function.

Like the memories 88, 96 described above, the memory 104 may be implemented using one or more memory devices such as solid state memory devices and/or other types of memory devices. Information that may be stored in the memory 104 includes software implementing the clinical interface module 106, information associated with a patient being monitored, sensor readings, alarm states, alarm thresholds, and/or possibly other information.

Various options for implementing the functions of a PMU, a server, and a CMP, in hardware, software, firmware, or some combination thereof, will be readily apparent to a skilled person from the foregoing, primarily functional, description of the example PMU 80, the example server 90, and the example CMP 100, and the descriptions of various methods according to embodiments of the invention. For example, those skilled in the art will be familiar with many types of sensors, communication protocols, processing elements, and user interfaces that may be implemented in conjunction with a PMU, a server, and a CMP.

FIG. 9 illustrates, in block diagram form, an alarm management apparatus 110 that may implement embodiments of the present invention. In a remote monitoring architecture, the apparatus 110 might be implemented at each CMP. However, it would also be possible to implement such an apparatus at some central location, such as at a server, or even at a PMU. In the latter case, both monitoring and alarm management are provided locally, at a patient site, and possibly within one physical unit. Local alarm processing might be preferred, for example, when communication with a patient site is unreliable.

In the example apparatus 110, a user interface 112 is operatively coupled to an alarm criterion settings store 114 in a memory 113 and to an analysis module 118. The analysis module 118 is operatively coupled to the alarm criterion settings store 114, to a persistence history store 116 in the memory 113, and to one or more sensor interfaces 119.

The user interface 112 and the sensor interface 119 may be the same as the user interface 108 and the sensor interface 86 described above with reference to FIG. 8 and FIG. 6, although in the preceding Figures these interfaces are provided in different equipment. It should be appreciated, however, that the interfaces 112, 119 are not intended to be restricted to interfaces that provide for only local transfer of information.

Alarms generated by the analysis module 118 could be reported locally to a user through a user interface 112 such as a monitor, but could also or instead be transmitted to a remote system for reporting to a user. User inputs could similarly be received from a local user or a remote user. The sensor interface component 119 in FIG. 9 could similarly include one or more interfaces for receiving readings from locally deployed sensors and/or from remotely located sensors.

Therefore, in the apparatus 110, the user interface 112 and/or the sensor interface 119 could include a network interface or other interface that provides for any or all of: remote user input, remote alarm reporting, and remote sensor reading collection.

Depending on the amount of information to be stored, the memory 113 could include one or more memory devices for storing the alarm criterion settings 114 and the persistence history 116. Solid state memory devices and/or other types of memory devices may be used for this purpose. Since combination alarms and alarm persistence according to embodiments of the invention are not interdependent, some implementations could include only the alarm criterion settings store 114 for combination alarms or the persistence history store 116 for persistent alarms. Although not specifically shown in FIG. 9, an apparatus according to an embodiment of the invention might also store other information in the memory 113, such as software implementing the analysis module 118, sensor readings, alarm states, etc. Such additional information could be stored in the same memory device(s) as the alarm criterion settings store 114 and/or the persistence history store 116, or in one or more further memory devices.

The analysis module 118 may be implemented in hardware, software, firmware, or combinations thereof, using one or more of the processing elements described above for instance, and is therefore described below primarily in terms of function. Based on this functional description, a person skilled in the art would be enabled to implement embodiments of the invention in any of various ways.

As noted above, the sensor interface 119 enables the apparatus 110 to receive respective values for multiple physiological conditions from sensors, which may include local sensors which are co-located with the apparatus and/or remotely located sensors. The analysis module 118 determines whether the received values satisfy respective alarm criteria, stored in the alarm criterion settings store 114, for the physiological conditions, and if so, raises an alarm. User inputs for configuring the respective alarm criteria in the alarm criterion settings store 114 can be received through the user interface 112.

The analysis module 118 may also support individual vital or sensor alarms. To this end, the analysis module 118 further determines whether an individual alarm criterion, also stored in the alarm criterion settings store 114, for one of the physiological conditions is satisfied by the received value of that physiological condition. If so, the analysis module 118 raises an individual alarm for that physiological condition.

Not all received sensor reading need necessarily be used for combination alarm processing. A value for another physiological condition may be received through the sensor interface 119 and used by the analysis module 118 to determine whether an alarm criterion for the other physiological condition is satisfied. An individual alarm for that other physiological condition is raised where the received value for the other physiological condition is satisfied.

As noted above, the analysis module 118 may process received sensor readings for combination alarms, individual vital or sensor alarms, or both.

The analysis module 118 may also or instead support persistent alarms. In this case, values of a physiological condition are periodically received from a sensor through the sensor interface 119, and the analysis module 118 determines whether the received values have satisfied an alarm criterion for at least a predetermined portion of a predetermined period of time. If so, the analysis module 118 maintains an alarm.

Values received through the sensor interface 119 might indicate values of the physiological condition at respective times, as shown in FIG. 5, for example. The analysis module 118 can then determine whether the received values have satisfied the alarm criterion for at least the predetermined portion of the predetermined period of time by determining whether at least a minimum number of the received values that indicate values of the physiological condition during a time period equal to the predetermined period of time immediately preceding a time of a most recently received value satisfied the alarm criterion.

Alarm persistence for combination alarms may be provided where values of a multiple physiological conditions are received through the sensor interface 119. The analysis module 118 in this case determines whether the received values of the multiple physiological conditions have satisfied respective alarm criteria for at least the predetermined portion of the predetermined period of time and if so, maintains an alarm.

In some embodiments, the user interface 119 enables interaction with a user through a GUI. Alarms may thus be raised and/or maintained in the form of an alarm indication in the GUI.

What has been described is merely illustrative of the application of the principles of the invention. Other arrangements and methods can be implemented by those skilled in the art without departing from the spirit and scope of the present invention.

1. A method comprising:
periodically receiving values of a physiological condition from a sensor;
determining whether the received values have satisfied an alarm criterion for at least a predetermined portion of a predetermined period of time; and
maintaining an alarm where the received values have satisfied the alarm criterion for at least the predetermined portion of the predetermined period of time.

2. The method of claim 1, wherein the received values indicate values of the physiological condition at respective times, and wherein determining comprises determining whether at least a minimum number of the received values that indicate values of the physiological condition during a time period equal to the predetermined period of time immediately preceding a time of a most recently received value satisfied the alarm criterion.

3. The method of claim 1, further comprising:
periodically receiving values of a further physiological condition from a further sensor,
wherein determining further comprises determining whether the received values of the further physiological condition have satisfied a further alarm criterion for at least the predetermined portion of the predetermined period of time, and
wherein maintaining comprises maintaining the alarm where both the received values of the physiological condition have satisfied the alarm criterion for at least the predetermined portion of the predetermined period of time and the received values of the further physiological condition have satisfied the further alarm criterion for at least the predetermined portion of the predetermined period of time.

4. The method of claim 1, wherein maintaining comprises maintaining an indication of the alarm in a GUI (Graphical User Interface).

5. A computer readable medium having computer readable instructions stored thereon that when executed implement the method according to claim 1.

6. Apparatus comprising:
a sensor interface that enables the apparatus to periodically receive values of a physiological condition from a sensor; and
an analysis module, operatively coupled to the sensor interface, that determines whether the received values have satisfied an alarm criterion for at least a predetermined portion of a predetermined period of time, and maintains an alarm where the received values have satisfied the alarm criterion for at least the predetermined portion of the predetermined period of time.

7. The apparatus of claim 6, wherein the received values indicate values of the physiological condition at respective times, and wherein the analysis module determines whether the received values have satisfied the alarm criterion for at least the predetermined portion of the predetermined period of time by determining whether at least a minimum number of the received values that indicate values of the physiological condition during a time period equal to the predetermined period of time immediately preceding a time of a most recently received value satisfied the alarm criterion.

8. The apparatus of claim 6, wherein the sensor interface further enables the apparatus to periodically receive values of a further physiological condition from a further sensor, and wherein the analysis module further determines whether the received values of the further physiological condition have satisfied a further alarm criterion for at least the predetermined portion of the predetermined period of time and maintains the alarm where both the received values of the physiological condition have satisfied the alarm criterion for at least the predetermined portion of the predetermined period of time and the received values of the further physiological condition have satisfied the further alarm criterion for at least the predetermined portion of the predetermined period of time.

9. The apparatus of claim 6, further comprising:
a user interface, operatively coupled to the analysis module, that enables interaction with a user through a GUI (Graphical User Interface),
wherein the analysis module maintains the alarm by maintaining an indication of the alarm in the GUI.

10. The apparatus of claim 6, wherein the sensor is located remotely from the apparatus.

11. A method comprising:
receiving respective values for a plurality of physiological conditions;
determining whether the received values satisfy respective alarm criteria for the plurality of physiological conditions; and
raising an alarm where the received values satisfy the respective alarm criteria for the plurality of physiological conditions.

12. The method of claim 11, further comprising:
receiving user inputs for configuring the respective alarm criteria for the plurality of physiological conditions.

13. The method of claim 11, further comprising:
determining whether an individual alarm criterion for one of the plurality of physiological conditions is satisfied by the received value of the one of the physiological conditions; and
raising an individual alarm for the one of the physiological conditions where the individual alarm criterion for the one of the physiological conditions is satisfied by the received value of the one of the physiological conditions.

14. The method of claim 11, further comprising:
receiving a further value for a further physiological condition other than the plurality of physiological conditions;
determining whether the received further value satisfies a further alarm criterion for the further physiological condition; and
raising an alarm for the further physiological condition where the received further value satisfies the further alarm criterion for the further physiological condition.

15. A computer readable medium having computer readable instructions stored thereon that when executed implement the method of claim 11.

16. Apparatus comprising:
a sensor interface that enables the apparatus to receive respective values for a plurality of physiological conditions; and
an analysis module, operatively coupled to the sensor interface, that determines whether the received values satisfy respective alarm criteria for the plurality of physiological conditions, and raises an alarm where the received values satisfy the respective alarm criteria for the plurality of physiological conditions.

17. The apparatus of claim 16, further comprising:
a user interface, operatively coupled to the analysis module, that enables the apparatus to receive user inputs for configuring the respective alarm criteria for the plurality of physiological conditions.

18. The apparatus of claim 16, wherein the analysis module further determines whether an individual alarm criterion for one of the plurality of physiological conditions is satisfied by the received value of the one of the physiological conditions, and raises an individual alarm for the one of the physiological conditions where the individual alarm criterion for the one of the physiological conditions is satisfied by the received value of the one of the physiological conditions.

19. The apparatus of claim 16, wherein the sensor interface further enables the apparatus to receive a further value for a further physiological condition other than the plurality of physiological conditions, and wherein the analysis module determines whether the received further value satisfies a further alarm criterion for the further physiological condition, and raises an alarm for the further physiological condition where the received further value satisfies the further alarm criterion for the further physiological condition.

20. The apparatus of claim 16, further comprising:
a memory for storing the respective alarm criteria for the plurality of physiological conditions,
wherein the analysis module accesses the memory to determine whether the received values satisfy the respective alarm criteria for the plurality of physiological conditions.
